(19) United States
(12) Patent Application Publication
(10) Pub. No.: US 2003/0083602 A1
(43) Pub. Date: May 1, 2003

(54) Flexible joint protection device

(76) Inventor: Peter D. Haaland, Louisville, CO (US)

Correspondence Address: SHERIDAN ROSS PC, 1560 BROADWAY, SUITE 1200, DENVER, CO, 80202

(21) Appl. No.: 10/003,567

(22) Filed: Nov. 1, 2001

(51) Int. Cl. A61F 5/00

(52) U.S. Cl. 602/16; 602/5

(57) ABSTRACT

A joint support device is disclosed that protects a joint from hyperextension. The device allows substantially unrestricted joint movement within a first range of joint movements, which is within a normal range of joint movement. However, when there is joint flexure beyond the first range, the device resists such flexure. In particular, the device can progressively increase its resistance with increased joint flexure in a second range of joint movement until a predetermined upper limit on the flexure is reached wherein substantially no further joint flexure is possible. Such an upper limit may be near but not at a flexure of the joint that could cause hyperextension. The device includes rigid elements that attach on opposite sides of the joint, and includes extensible assemblies extending between the rigid elements for providing the functionality recited above. Such an assembly may be columns of spacers threaded together with an extensible cable, or a plurality of chains having links with a high a tensile modulus and high failure stress.

### FIELD OF THE INVENTION

The invention relates to injuries caused by hyperextension of joints including damage to ligaments, tendons, muscle, and bone near the wrist, elbow, shoulder, hip, knee, and ankle. In particular, the invention describes a device that can reduce the injury caused by falls or other accidental events while participating in sports or physical work. The invention also relates to rehabilitation of persons recovering from joint injuries, surgery, or medical conditions that reduce joint function.

### BACKGROUND OF THE INVENTION

Injuries to joints are a common result of accidents, and in particular accidents in sports. For example, of the 106,000 persons injured while in-line skating in 1996, 49.6% involved the wrist, elbow, knee, ankle, or shoulder joints. Wrists alone accounted for one quarter of these injuries, according to the U.S. Consumer Product Safety Commission. Acute injuries caused by hyperextension of the wrist are also common in snowboarding, football, volleyball, hockey, and other sports where falls are cushioned by an extended arm and hand.

Common experience shows that the motion of two body parts that are connected by a joint occurs with minimal effort and no damage over a range of angles that vary with the joint location (i.e. ankle, knee, shoulder, etc.) and also with a person's general health. It is further commonly recognized that extension beyond this normal range can cause a variety of injuries including bruising, tearing, or rupture of ligaments, cartilage, tendons, nerves, vasculature, and bones. Prior art joint protective devices provide some protection against these injuries but limit the range of motion around the joint, in many cases resulting in degraded biophysical performance. The present invention protects joints from hyperextensive injuries while minimizing performance degradation by permitting normal motion with minimal restriction.

Static wrist protection devices have been invented to mitigate the impact of these injuries. For example, Levine describes a snowboard wrist protector with a rigid element in U.S. Pat. No. 5,303,667, which is incorporated herein by reference. This device has the disadvantage that the rigid element restricts the user to a small fraction of the normal range of joint motion.

Another rigid wrist guard integrated with a glove is disclosed by Dorr in U.S. Pat. No. 5,537,692 and is also incorporated herein by reference. As with the Levine device, this wrist guard limits the range of the users' wrist motion. This is a particular disadvantage for competitive snowboarding, where wrist flexibility is required to execute aerobatic maneuvers such as the tail grab, methods, mute grab, nose grab, inverted moves, and other maneuvers familiar to those practiced in the art of snowboarding.

A hinged wrist guard built from two rigid elements improves the range of motion that is afforded to a wearer and is disclosed by Oetting et al. in U.S. Pat. No. 5,778,449, which is incorporated herein by reference. This device, which permits some flexion in the directions normal to the plane of the palm, does not permit the normal range of motion in the direction parallel to the plane of the palm. Another example of prior art for a sports protective glove is disclosed by Morrow and Roberts in U.S. Pat. No. 5,983,396 and is incorporated herein by reference. The Morrow device offers improved padding and adjustment of the location of a rigid wrist guard, but restricts the range of wrist motion as do the other prior art devices cited above.

The normal range of joint motion is well established in the medical literature. A study of the wrist is described by Nelson et al. in Wrist Range of Motion in Activities of Daily Living, (Advances in the Biomechanics of the Hand and Wrist, (ed. F. Shuind et al., Plenum Press, NY, 1994, p. 329-334), which is incorporated herein by reference. FIG. 1 is a reprint of Table 2 from this paper, where the routine excursion of the wrist is seen to vary from +11 to −40 degrees in the plane of the palm and +49.8 to −51.1 degrees perpendicular to the plane of the palm. The data in FIG. 1 were acquired for activities of normal living such as combing hair, using a telephone, turning a faucet, and the like. As will be appreciated by those practiced in the art of biomechanics, this range can be extended somewhat in sporting activities; for example the range of wrist flexion in a tennis serve or golf swing may exceed the bounds defined for the daily living activities described by Nelson et al. The normal range of motion for a healthy joint may also be lessened as it heals from injury, surgery, or during physical therapy. In any case, extension well beyond this normal range will result in injury to the joint tissues including sprains, tears, and fractures of ligament, tendon, bone, and muscle.

The problem, therefore, is to provide protection from, e.g., hyperextension of the joint while minimally limiting the normal range of joint motion.

### SUMMARY OF THE INVENTION

The present invention is a joint support/protective device (herein simply denoted a “joint support”), wherein variable resistance or support is provided to an adjacent joint according to a bending or flexing of the joint. In particular, the joint support of the present invention is configured to provide minimal resistance to a bending or flexing motion of an adjacent joint whenever such motion is within a predetermined range that is preferably the normal range of motion for the joint. However, as the joint motion approaches the limits of the normal, or generally predetermined, range of the joint, the joint support stiffens, inducing a redistribution of the load or force on the joint so that, e.g., the joint is not subjected to hyperextension.

Other features and benefits of the present invention will be evident from the Detailed Description hereinbelow and the accompanying drawings.

### BRIEF DESCRIPTION OF THE DRAWINGS

FIG. 1 is a table that summarizes the range of wrist motion (degrees) for a series of activities that are representative of normal living taken from Nelson et al. (loc. cit.).

FIG. 2 is a cross-sectional schematic of one embodiment of the present invention. A contoured plate (210) is connected by a cable (260) that passes through one or more spacer rings (230) and a second rigid element (220) to an anchor or cable stop (250) that may be separated from the base by a coiled spring or other elastically deformable element (240).

FIG. 3A is an expanded view of two spacer elements (230) from the previous FIG. 2. The geometric relationships between the radial spacing (305), the angle (315), and the length of cable that spans the gap when the device is flexed (325) are illustrated.

FIG. 3B is a cross sectional view of another embodiment of two spacer elements 230 (also denoted disks herein), wherein a central core 332 made from a high elastic modulus material (e.g. steel) and a deformable, low elastic modulus covering 331 (e.g. rubber).

FIG. 4 is a drawing of several possible configurations for the spacer rings that differ in the constraints that they impose on flexure before fully tensing the cable 260.

FIG. 5 is a plot of the locus and envelope (in degrees) for normal wrist motion of one subject and is reproduced from Salvia et al. (Advances in the Biomechanics of the Hand and Wrist, F. Schuind et al. eds., Plenum Press, New York, 1994, p. 313-327)

FIGS. 6A through 6E show illustrations of two additional embodiments. FIGS. 6A through 6C shows a first embodiment of the present invention that employs interlocking elements 608 forming a single chain 260. In particular, the FIGS. 6A through 6C show the flexing of the chain 260 as the angle 652 between the rigid elements 610 which, e.g., may fit over and/or around a user's limb on each side of a joint being protected by the present embodiment. Note that the two rigid elements 610 are coupled together by the chain 620 of interlocking links 608. FIG. 6A shows the present embodiment in an unflexed or equilibrium state with an expanded view 640 of the link geometry shown in the lower portion of FIG. 6A. FIG. 6B illustrates partial flexion of this first embodiment so that the length between the two chain connection points 630 (also denoted anchors) is equal to the sum of the link 608 diameters. An expanded view 660 of the link geometry is shown in the lower portion of FIG. 6B. FIG. 6C shows this first embodiment nearing hyperextension (of a joint at separation 618), where the links 608 are fully deformed and the mechanical load is carried by the tension of the material(s) from which the links 608 are composed. An expanded view 613 of the link geometry is shown in the lower portion of FIG. 6C. FIGS. 6D and 6E show a second embodiment of the present invention that employs interlocking elements 608 forming a plurality of chains 260. In particular, FIG. 6D shows a side view of this second embodiment, wherein the chains 260 are positioned about rigid elements 610. FIG. 6E shows a top view of the second embodiment of FIG. 6D. Note that the rigid elements 610 are shown as oval shaped cylinders wherein, e.g., an arm may be inserted therein.

FIG. 7 shows an embodiment whereby the variable resistance to flexure is provided by a stack of mating spacers 720 and 710 whose shapes differ. Cylindrical spacers 720 are stacked alternately with spherical spacers 710, as seen in an exploded view 730. The spherical spacers 710 fit in opposing spherical depressions 715 as shown in view 740. A cable 260 is threaded through the spacer elements and optionally a compressible element 240 and affixed to rigid elements as in FIG. 2 (not shown in FIG. 7). Minimal, primarily frictional resistance to motion is produced during joint flexure until the faces of the cylindrical spacers come into contact with each other, as shown in the rightmost view 750 of the present figure.

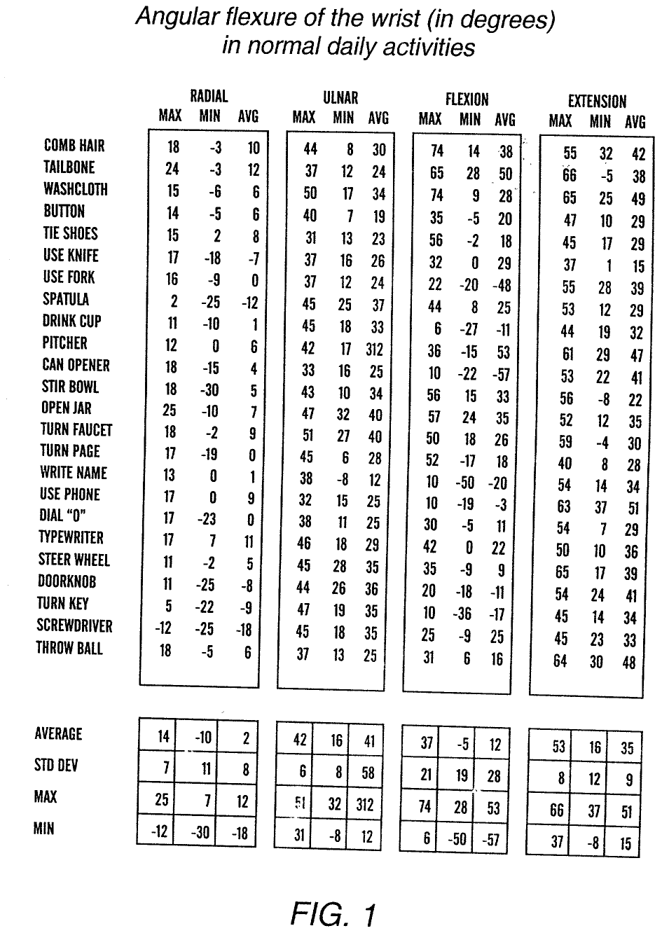

### DETAILED DESCRIPTION OF THE INVENTION

The central element of the device according to the present invention is a structural component that has variable stiffness. In other words, the element is flexed with minimal or no effort over a range corresponding to the normal range of joint motion and requires substantial effort to flex beyond the normal range.

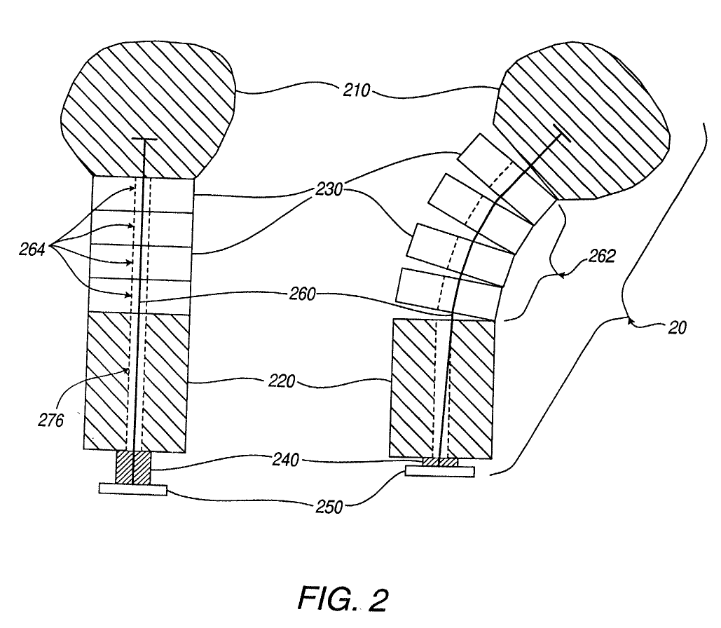

One embodiment of the joint support 20 of the present invention that provides protection to, e.g., the wrist joint (or other body joint) of a user is schematically shown in FIG. 2 in a straightened orientation on the left and a bent orientation on the right. A first rigid element 210 is shaped to fit a first body part of a user. For example, the body part may be the palm of a user's hand. The first element 210 is designed to be affixed to the first body part by for example a strap (e.g., Velcro strap), glove, or woven cloth or fabric sleeve (not shown). Alternatively, the first element 210 may be molded in such way that the body part may fit within a molded portion for thereby securing the first element to the body part.

The first element 210 is connected to a cable 260 that has high elastic tensile modulus and failure strength. For example, the cable 260 may be made of one or more of the following materials: steel, titanium, fiberglass-graphite composite, or high strength organic polymer (polyaramide, e.g., Kevlar™, Kapton™, rigid rod liquid crystals, e.g., p-paraphenylene benzo-bisthiazole or bisoxazole). Moreover, note that the cable is attached to the first element 210 by any number techniques, including welding, epoxy bonding, compression fitting, threading, or soldering.

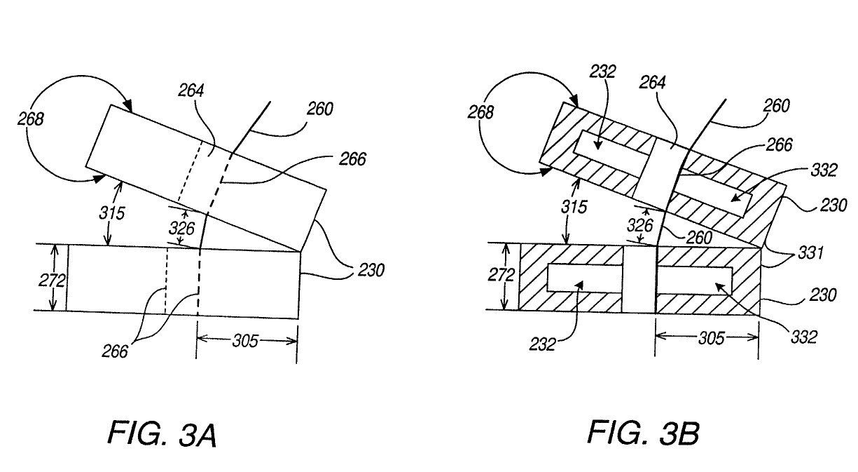

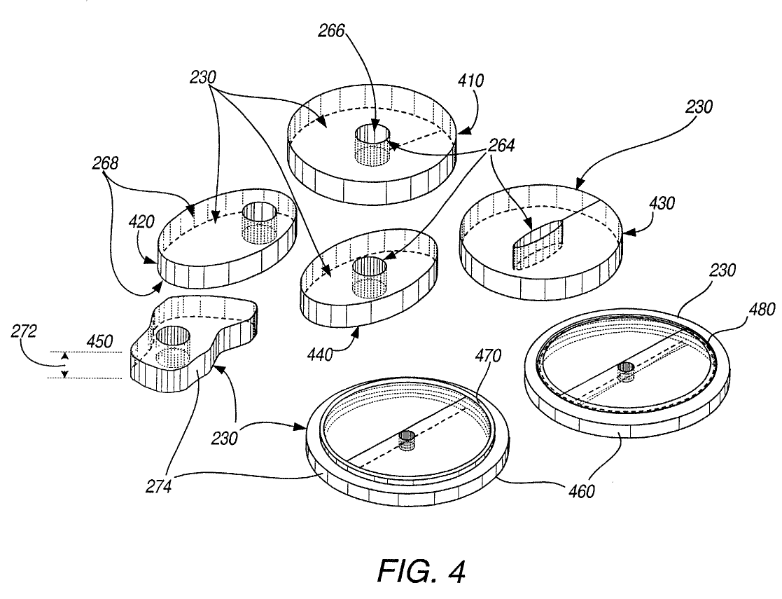

Referring to FIGS. 2, 3A, 3B, and 4, the cable 260 is threaded through a column 262 of one or more spacer disks 230. In particular, the cable 260 threads through an opening 264 (traversing between opposed sides 268) in each of one or more spacer disks 230. At least some embodiments of the spacer disks 230 are best shown in FIG. 4. Note that such spacer disks 230 can have various shapes, and the openings 264 can also be variously shaped as will be further described hereinbelow. Moreover, although not shown here, such spacer disks 230 may also vary in thickness 272, and in fact, the thickness may vary within a single spacer disk 230 so that, e.g., the outer perimeter (e.g., perimeters 274) of the spacer disk can vary in thickness. Additionally, note that although the internal walls (e.g., FIGS. 3 and 4) are shown as being cylindrical in shape, it is within the scope of the present invention that other shapes may also may utilized therefor such as conical shaped openings, or, horn or hour glass shaped curved internal walls 266. The spacer disks 230 may be made of materials with high compressive moduli and high failure strengths such as of one or more of the following materials: metals such as steel, titanium, aluminum; metal-matrix composites; ceramics such as aluminum oxide, boron nitride, and titanium oxide; polymers such as polycarbonates, polyacrylates, polyimides, or poly-dicyclopentadiene.

In the embodiment of FIG. 3B, the spacer disks 230 are comprised of materials that have elastic compliance that varies with flexure. Thus, the spacer disks 230 of FIG. 3B may be composed of materials that have a high modulus (stiff) core 332 surrounded by a lower modulus coating 331 as shown in FIG. 3B. Accordingly as the cable 260 is tensed, e.g. to within 10% of the normal range of flexure, compression of the deformable coating 331 resists flexure with less force than would the case with monolithic stiff disks 230 composed of a single material. Ultimately during flexure, the coating 331 reaches the limit of its elastic deformation, beyond which further flexure is resisted by the full support provided by the stiff core 332. As is apparent to those practiced in the art of mechanical engineering, the composition, elastic modulus, and thickness of the coating 331 as well as the shape, elastic modulus, and composition of the stiff core 332 will determine the precise relationship between flexure and mechanical compliance of the joint support.

As will be apparent to those practiced in the art of mechanical engineering, the materials from which the spacers are formed are chosen to bear the load that might otherwise lead to hyperextension of the joint. The mechanical properties of bone are understood by those practiced in the art of orthopedics. For example, the tensile, compressive, and shear moduli of bone are 17.9, 4.9, and 3.9 GPa, respectively. Collagen and elastin have elastic moduli of about 1 GPa, and 0.6 GPa, respectively. The stress-strain characteristics of a joint depend on these properties as well as the detailed shape and composition of the joint elements. The load borne by the joint support also depends on both the stiffness (modulus) and geometry of its components according to the laws of classical mechanics. According to the present invention, the composition and geometry of the flexible column 262 are sufficiently stiff (i.e., have high enough moduli) to force the load borne by the joint support to substantially exceed that borne by the protected joint itself.

In the embodiment of the present invention shown in FIG. 2, the cable 260 is also threaded through an opening 276 (FIG. 2) in a second rigid element 220 that is shaped to fit a second body part, wherein there is a skeletal joint joining the first and second body parts: such pairs of first and second body parts being, e.g.: (a) wrist and forearm, (b) forearm and upper arm (above the elbow), (c) upper and lower leg, (d) consecutive segments of a finger, or (e) ankle and lower leg. Thus for example, if the first element 260 is secured to the user's wrist, then the second element 220 may be secured to the user's forearm by a strap, the second element may be molded so that the user's forearm securely fits within the mold. Thus, the second element 220 may be contoured to comfortably fit the shape of the second body part. The second element 220 may be secured to the second body part by any of the techniques for securing the first element 210 to the first body part, and second element may be made from substantially the same materials as the first element 210.

Still referring to FIG. 2, a portion of the cable 260 extends through the channel 276 and is attached to a cable stop 250 that is unable to fit through the channel 276. Between the second element 220 and the cable stop 250, there may be an elastically deformable element 240 (e.g., a spring or compressible elastomer) for asserting a tension on the cable 260. Moreover, note that the deformable element 240 is also too large to fit through the channel 276. Further note that the cable 260 is able to shift a predetermined amount within the canal comprising the openings 264 and the channel 276 so that there is a prescribed amount of bending of the column 262 without the cable 260 stretching and without (any) deformable element 240 compressing. In particular, the range of bending of the column 262 in any direction, prior to an opposing force being exerted by a stretching of the cable 260 and/or a compression of (any) deformable element 240, depends on at least:

(a) the extent(s) of the cross section(s) of each opening 264 in the direction of bending (more generally, the extent and shape of the internal walls 266 of each opening 264 where the cable 260 may contact the internal walls during bending); e.g., the greater such extents are in a given direction, the greater amount of substantially unconstrained bending in that direction;

(b) the extent(s) from the internal wall 266 to the perimeter 274 of each spacer disk 230 in the direction of bending (more generally, the extent to and the shape of the perimeter 274, including thickness 272) where adjacent spacer disks 230 contact one another).

As mentioned above, the rigid structural elements 210 and 220 can be fixed to each side of a skeletal joint by, e.g., fabric, Velcro straps. Optionally, one or more of the rigid elements 210 and 220 and/or the column 262 may be integrated into a body part fitting covering such as a glove, a sleeve, a bandage, or a brace. For example, the flexible column 262 of one or more spacer disks 230 (threaded by the cable 260) may be surrounded by a flexible sleeve such as a plastic tube to prevent pinching of the skin when the column 262 bends with the adjacent skeletal joint of the user that is between the first and second elements, 210 and 220 respectively as shown in FIG. 2.

In operation, when the joint support 20 of the present invention is properly secured adjacent to a user's joint that is to be supported, the column 262 is able to flex subject only to the frictional forces and the cable tensioning force of the (any) deformable element 240 until the deformable element is fully compressed and the cable stop 250 engages with the end of the rigid element 220. Further bending loads placed upon the joint support 20 increases the tension of the cable 260, and accordingly increases the stiffness of the column 262. As will be recognized by those practiced in the art of mechanics, tension on the cable 260 is accompanied by compressive stress at the exterior edge of the spacers 230. The combined effect of tension in the cable and compression of the spacers provides mechanical resistance to further flexure. In at least some embodiments of the joint support 20, the stiffness of the column 262 is directly proportional to the elastic modulus of the cable 260.

The flexure of the column 262 at which the joint support 20 stiffens is best understood with reference to FIG. 3, which shows a schematic expanded view of two adjacent ones of the spacer disks 230 that are angularly skewed relative to one another. Flexure of these spacer disks 230 increases the length of cable 260 by an amount s (identified by label 325. FIG. 3) that depends on: (i) the angle of flexure a (label 315, FIG. 3), and (ii) the distance r (label 305, FIG. 3) from the perimeter 274 to the portion of internal wall 266 in contact with the cable 260 according to the following trigonometry equation:

s=2 r sin(a/2). (Equation 1)

Flexure (e.g., angular skewing of the spacer disks 230 of FIG. 3) is impeded only by friction, plus, the tension supplied by compression of the (any) deformable element 240. The amount of flexion prior to the spacer disks 230 of FIG. 3 (or more generally, the column 262) being substantially prevented from further relative skewing/bending is dependent upon the total length of cable 260 that can be made available for extending between the first and second rigid elements 210 and 220 when the deformable element 240 (if present) is fully compressed by the cable stop 250 and/or the cable stop engages the rigid element 220.

If each of the spacer disks 230 is cylindrical and its cable opening 264 is also cylindrical and centered within the spacer disk, then the angle a (315) will be the same in all directions for a fixed amount of exposed cable s (325). An example of a symmetrical spacer disk 230 is shown in FIG. 4, wherein the disk is also labeled 410. However, as is apparent from consideration of Equation (1) above, the shape of a spacer disk 230 and the location of the hole through which the cable 260 traverses also permits the angle a to vary when the spacer disks are skewed relative to one another in a different direction. Moreover, the angle a may vary according to the skewing/bending direction for the same amount of exposed cable s (325); because r (305) and s (325) determine a (315); i.e.,

a=2sin⁻¹(s/2r), 0<r<s/2. (Equation 2)

One example of a spacer disk 230 that allows the angle a to vary with the direction of skewing is the elliptical spacer disk labeled 440 in FIG. 4. Note that since this spacer disk has its opening 264 at its center, this will provide flexure that is symmetrical along each of the semi-axes of the ellipse, with the smallest range of a corresponding to the semi-major axis and the largest range corresponding to the semi-minor axis of the opening. FIG. 4 also shows an elliptical spacer disk 420 with a non-centered opening 264. This spacer disk 420 may allow flexure in different directions to change inversely with r for that direction. Note also that the opening 262 may have a shape other than circular, for example, spacer disk 430 (FIG. 4) has an elliptical shaped opening for varying the distance r with skewing/bending direction.

In at least some embodiments of the spacer disk 230, these disks have more features than heretofore have been described. In particular, it is desirable for the spacer disks 230 to skew/bend relative to one another smoothly and predictably. Thus, instead of adjacent spacer disks 230 having substantially planar opposed sides 268, such disks may have mating portions that assure smooth, repeatable, predictable stiffening of the joint support 20 at predetermined orientations. Such mating portions can inhibit unintended lateral motion between the spacer disks 230 such as two adjacent such disks sliding relative to one another along their sides 268 which are in contact. Note that such a sliding motion is, in general undesirable in that it lessens (if not substantially disables) the stiffening effect of the joint support 20 when such stiffening is desired at predetermined orientations. In particular, the spacer disks 230 may have mating ridges and grooves so that when the cable exerts force against the internal walls 266 of the openings 264, an induced sliding force can be constrained by on the facing sides 268 of the spacer disks 230. Opposing faces of disk elements 460 are shown in FIG. 4, with a circular convex ridge feature 470 shaped to align with a corresponding groove 480. The geometric shapes of the ridge and groove are shown as circular for clarity in the figure and may clearly take on any geometric shape so as to constrain the lateral motion of the spacer disks.

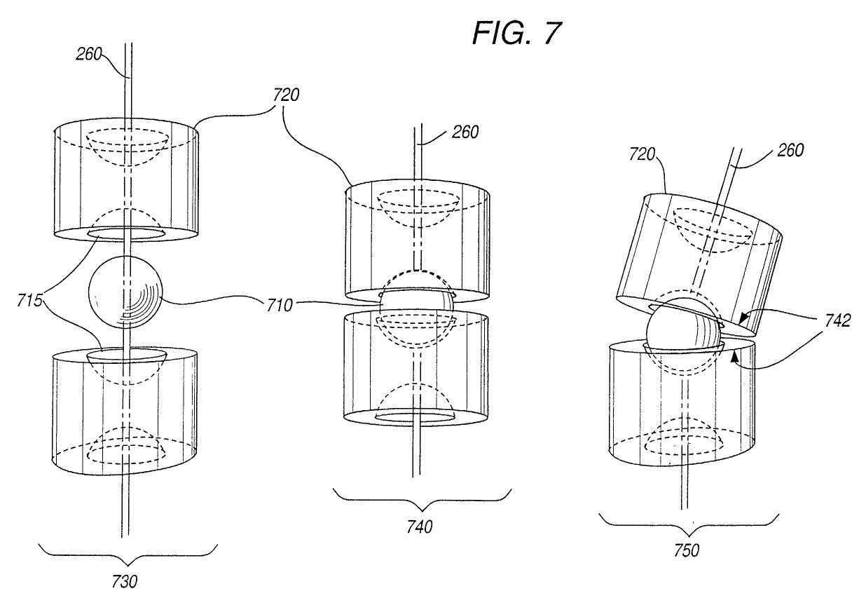

The spacer disks need not, according to the present invention, have identical shapes. FIG. 7 illustrates two types of spacer disks 230 in an arrangement that provides minimal flexural resistance over a predetermined range and maximal resistance outside of the predetermined range. Cylindrical spacer disks 230 (herein labeled 720) are constructed with spherical depressions 715, which have the same radius of curvature as a spherical spacer disk 230 (herein labeled 710). The spacer disks are aligned by a central cable 260 and connections to rigid bodies (e.g., elements 210 and 220 not shown in FIG. 7) as described previously with reference to FIGS. 2 through 4. Mechanical resistance to flexion is limited to friction between the spacer disks 710 and 720 when the edges of spacer disks 720 do not touch each other as shown in the middle view 740 of FIG. 7. As the alignment of the spacer disks 720 changes with respect to one other due to a flexing of the adjacent joint that is being protected, the edges 742 of consecutive spacer disks 720 eventually touch, as shown in the rightmost view 750 of FIG. 7. Further flexure is thereafter constrained by the force required to compress the spacer disks 720 (and to some extent the spacer disks 710), the elasticity of the cable 260, and the compression characteristics of the (any) cable stop 250 (not shown in FIG. 7). Accordingly, the resistance to flexure varies in a predetermined way with the amount of flexure. As will be obvious to those practiced in the art of mechanical engineering, the shapes of the spacers 720 and 710 can be varied so that the range of flexure within which the resistance is minimal can be varied. In particular, the spacers 720 need not have a common uniform size and/or shape. For example, the height of the cylindrical disks 720 may vary from one these spacers to another. Additionally, circular ends of such a spacer 720 need not have the same diameters. Indeed, the edges 742 need not be circular at all. Furthermore, the spherical spacers 710 need not all have a common diameter.

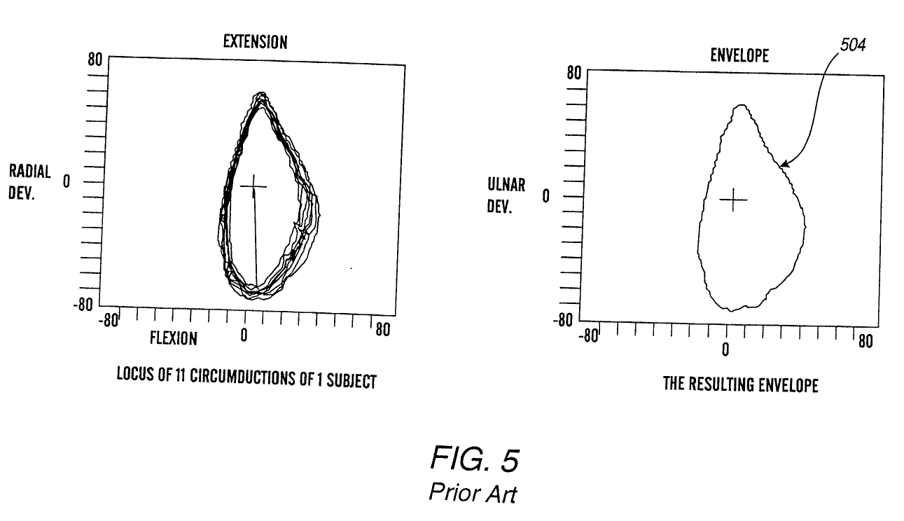

In a preferred embodiment of the invention the shape of the spacer elements 230 is chosen to give a range of joint flexure that is matched to the normal range of motion for the joint that is being protected. This range varies with the joint and among individuals for the same joint. The range is straightforwardly measured as described, for example, by Salvia et al. in The Envelope of Active Wrist Circumduction: An In-vivo Electrogoniometric Study (Advances in the Biomechanics of the Hand and Wrist, F. Schuind et al. eds., Plenum Press, New York, 1994, p. 313-327), and which is incorporated herein by reference. FIG. 5, which is reproduced from this reference, illustrates the asymmetry of the envelope 504 of normal wrist motion for one subject; i.e., ranges of wrist flexure represented by points on the right graph that are within the envelope 504 are generally considered within the normal range of wrist flexure. The angular motion of the palm with respect to the forearm in degrees shows motion parallel to the plane of the palm toward the thumb (Radial Deviation), away from the thumb (Ulnar Deviation), and perpendicular to the plane of the palm toward (Flexion) and away from (Extension) the palm's surface.

Moreover, the size, thickness, shape, and orientation of the cable channel (i.e., the collective set of openings 264, plus the channel 276, through which the cable 260 traverses between, e.g., the first element 210 and the second element 220) is determined so as to bring the cable 260 into full tension, that is, to engage the cable stop 250 with a fully compressed deformable element 240, when the flexure of the adjacent joint extends to the edge of an envelope such as the one shown on the right panel of FIG. 5.

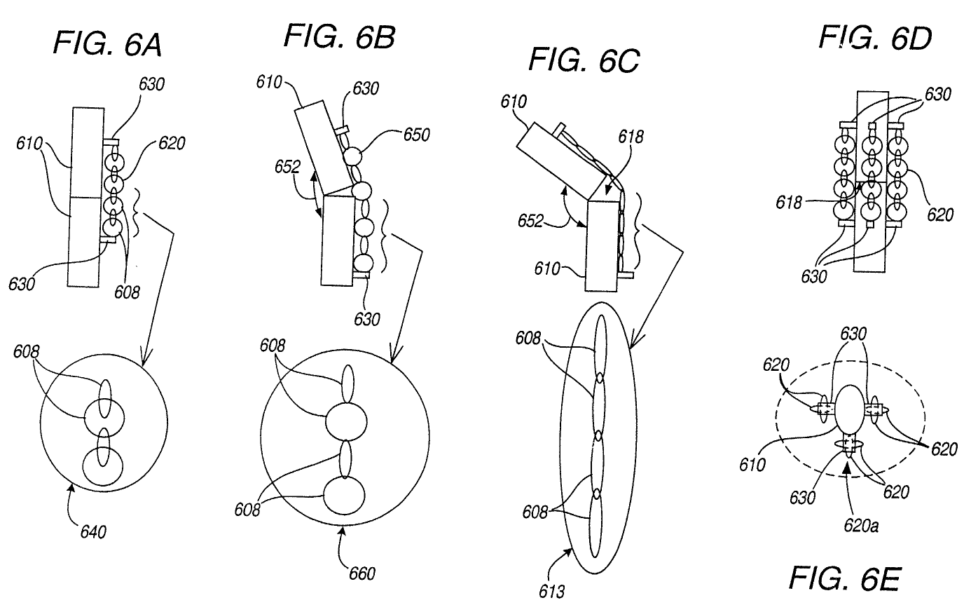

Two alternative embodiments of the invention are shown in FIGS. 6A through 6E. The first embodiment, shown in FIGS. 6A through 6C, uses interlocking links 608 (also denoted loops herein) to form a chain 620 for providing a resistance to flexure, in one direction; i.e., substantially within the plane of these figures by decreasing the angle 652. The single chain 620 has a low resistance in the normal range of angle 652 variations of joint motion but the chain stiffens as the joint approaches hyperextension (e.g., angle 652 goes below a predetermined limit). The rigid elements 610 attach to a user's body and may be located on opposing sides of a joint to be protected (which is not shown, but would be approximately located at the separation 618, FIG. 6C). The interlocking loops 608 forming the chain 620 are made from metal wire, polymer strands, carbon fiber, or other material (or composition of materials) with a high tensile modulus and high failure stress. The chain 620 is rigidly attached at each end to one of the rigid elements 610 by the anchors 630 (e.g., a fastener or an adhesive connection), such anchors may be composed of metal, or other materials with at least as high a tensile modulus and high failure stress as the chain attached thereto. When the joint adjacent the present embodiment is at its equilibrium, unflexed, or resting position the chain 620 provides low resistance to predetermined (non-damaging) angular joint deflections because the extension of the chain is less than the sum of the diameters of the loops as shown in view 640 (FIG. 6A). Note that there may be some small resistance due to friction between links 608; however, such resistance is inconsequential. Accordingly, as the joint is flexed further the links 608 of the chain 620 are tensed into contact while retaining their circular, or other predetermined shape (as shown schematically by the link 650, FIG. 6B) and in the expanded view 660 (FIG. 6B). Further additional flexure of the joint distorts the chain links 608 and requires a force that is approximately directly proportional to the rigidity or hoop strength of the individual links 608, both rigidity and strength being controlled by selection of the link material, size, and shape. As the flexure increases to the edge of the normal (more generally, predetermined) joint flexure envelope (along the angular range for angle 652), the chain links 608 become fully distorted by tension (as shown in FIG. 6C), so that the resistance to further flexure is approximately directly proportional to the high elastic modulus of the fiber from which the links 608 are manufactured. Note that the schematic presentation shown in FIGS. 6A through 6C shows a simplified embodiment of the present embodiment wherein only one chain 620 is shown. However, a plurality of such chains 620 maybe appropriately distributed about the joint to be protected so that a greater range of undesirable joint movements can be inhibited; e.g., simultaneously in a plurality of different planes of joint movement. In particular, the range of joint flexure in any direction can be adjusted by selecting the location of the chains 620, diameter of the links 608 and number of links 608 for the chains.

Accordingly, a second embodiment of the invention including a plurality of chains 620 is shown in FIGS. 6D and 6E. Note that each of the chains 620 and the other components in FIGS. 6D and 6E are substantially identical to the components in FIGS. 6A through 6C having identical labels. Thus, the chains 620 are fixed at their ends to the two separate rigid bodies 610, and the rigid bodies 610 are fixed to the protected joint on either side; e.g., by cloth, fiber composite, or plastic straps or sleeves. Additionally as above, number and diameter of chain links 608 in each chain 620 are selected to provide a low, primarily frictional, resistance to flexure when joint motion is within a pre-selected set of angular ranges. Beyond this first set of ranges further flexure is resisted, in a second predetermined set of angular ranges, by distortion of the shape of the chain links 608 according to the stiffness thereof. As the joint flexure is increased beyond the second set of angular ranges, a third set of angular ranges is entered that is defined, e.g., by a substantially maximal angular envelope that is allowable without hyperextension of the joint. Accordingly, in this third set of angular ranges, the links 608 are fully deformed and resistance to hyperextensive flexure is provided by tension of the links according to the elastic modulus and strength of the material from which they are made. Moreover, it is an aspect of the present invention that the plurality of chains 620 are oriented about the protected joint to restrict the flexure range differently in different directions of angular joint movement. Thus, referring to FIG. 6E, note that the positioning of the three chains 620 shown in this figure restrict the movement of a protected joint in each direction except the direction toward the chain 620a; i.e., the direction that does not cause an extension of any of the three chains 620. Moreover, by changing the extension characteristics of these chains and/or their positioning or corresponding anchors 630 about the rigid elements 610, different ranges or envelops of permissible joint movement can be provided while prohibiting joint movement beyond such an envelop.

It is also worth noting that although FIG. 6E shows the rigid elements 610 as having an oval shaped configuration within which, e.g., an arm or wrist maybe inserted, such rigid elements need not be a single unitary rigid component. Indeed, each of the rigid elements 610 may, e.g., include a plurality rigid strips having their longest extent (i.e., their length) extending generally parallelly with the chains 620, and wherein such strips are flexibly but securely attached to one another so that each of the rigid elements 610 can be wrapped about, e.g., an arm or wrist, and then secured thereabout with tape, Velcro straps, buckles, snaps, or other securing attachments.

The flexible joint protective of the present invention overcomes limitations to the range of motion that is inherent in prior art methods of joint protection. The invention embodiments described above, and particularly as illustrated in FIGS. 2 and 6, illustrate two embodiments that provide a graded resistance to flexure of a joint. Alternate embodiments that employ composite materials whose stiffness increases with bending strain or configurations that rely on strain dependent compression rather than tension are also encompassed by the present invention.

The joint protective device described herein has been illustrated primarily for protection of the wrist. However, as will be clear to those practiced in the art of orthopedics, the invention is equally suited to protection of the ankle, knee, hip, elbow, back, neck, and shoulder joints with suitable modification of the means for fixing the rigid elements (e.g., 610, 210) to the body on opposite sides of the joint.

The foregoing discussion of the invention has been presented for purposes of illustration and description. Further, the description is not intended to limit the invention to the form disclosed herein. Consequently, variation and modification commensurate with the above teachings, within the skill and knowledge of the relevant art, are within the scope of the present invention. The embodiment described hereinabove is further intended to explain the best mode presently known of practicing the invention and to enable others skilled in the art to utilize the invention as such, or in other embodiments, and with the various modifications required by their particular application or uses of the invention.

What is claimed is:

1. A flexible joint protective device comprised of:
(a) at least a first and second rigid element, wherein each of the first and second rigid elements is attached to a different side of a joint;
(b) one or more extendable assemblies that extend from said first rigid element to said second rigid element, wherein first and second distal ends of each of said assemblies are attached to said first and second rigid elements, respectively;
(c) wherein for a first range of joint flexure, there is substantially no resistance to the flexure and wherein for at least a second range of joint flexure there is resistance for preventing flexure beyond the second range.

2. The flexible joint protective device of claim 1, wherein said first range is substantially a normal range of motion for the joint, and the second range extends beyond the first range.

3. The flexible joint protective device of claim 1, wherein the second range does not extend to a joint flexure indicative of joint hyperextension.

4. The device according to claim 1, wherein at least one of said assemblies is comprised of
(a) one or more disks of a stiff material having an opening therethrough; and
(b) a cable for connecting said disks together, said cable threaded through the openings of said disks.

5. The device according to claim 4, wherein at least one of the first and second ranges are dependent on one or more of the following:
(a) a shape of at least one of the disks,
(b) a thickness of at least one of the disks,
(c) a composition of at least one of the disks
(d) a shape of the opening in at least one of the disks,
(e) a location of the opening in at least one of the disks,
(f) a length of the cable,
(g) a composition of the cable.

6. The device according to claim 1, wherein at least one of said assemblies includes a plurality chains, each chain having a plurality of links therein.

7. The device according to claim 6, wherein at least one of the first and second ranges are dependent on one or more of the following:
(a) a diameter of at least one of the links,
(b) a composition of at least one of the links,
(c) a number of links per chain,
(d) a number of the chains, and
(e) a location of chains around the joint.

8. The device according to claim 1, wherein the joint is selected from the group of joints: toe, ankle, knee, hip, finger, wrist, elbow, shoulder, back, and neck.
